(19) United States
(12) Patent Application Publication
(10) Pub. No.: US 2023/0347109 A1
(43) Pub. Date: Nov. 2, 2023

(54) Tool to Enable Catheter Insertion into a Mating Component

(71) Applicant: Bard Peripheral Vascular, Inc., Franklin Lakes, NJ (US)

(72) Inventors: Brian Zimmer, Salt Lake City, UT (US); Jonathan Darley, Sandy, UT (US)

(21) Appl. No.: 17/793,285

(22) PCT Filed: Jan. 15, 2021
(86) PCT No.: PCT/US21/13728

§ 371 Date: Jul. 15, 2022

Related U.S. Application Data
(60) Provisional application No. 62/962,012, filed on Jan. 16, 2020.

(51) Int. Cl. A61M 25/01; A61M 25/00; A61M 39/22

(52) U.S. Cl. CPC A61M 25/0133; A61M 25/0023; A61M 39/22

(57) ABSTRACT

Embodiments disclosed herein are directed to an insertion tool (100) for a catheter. The insertion tool (100) engages a proximal end of an introducer (10) and includes a guide channel (106) that aligns with a lumen of the introducer (10). The guide channel (106) supports the catheter (50) to maintain columnar integrity as the catheter (50) is urged distally, past a valve and into the lumen of the introducer (10). In an embodiment, the attachment structure can be inserted past the valve to define a pathway for the catheter. Embodiments also include an elongate opening to allow lateral ingress/egress of the catheter, a segmented cylindrical shape to provide a shortened elongate opening, and an offset elongate opening to allow a rolling introduction of the catheter to the guide channel to prevent abrasion of any coatings disposed on the catheter.

### PRIORITY

This application claims the benefit of priority to U.S. Provisional Application No. 62/962,012, filed Jan. 16, 2020, which is incorporated by reference in its entirety into this application.

### SUMMARY

Briefly summarized, embodiments disclosed herein are directed to an insertion tool for inserting a catheter into a lumen of an introducer. The insertion tool engages a proximal end of an introducer and includes a guide channel that aligns with a lumen of the introducer. The guide channel supports the catheter and maintains columnar integrity as the catheter is urged distally, past a valve, and into the lumen of the introducer. Embodiments include an attachment structure that is inserted past the valve to define a pathway for the catheter to pass through. The attachment structure can include an interference fit engagement with the introducer. Embodiments also include an elongate opening to allow transverse ingress/egress of the catheter, a segmented cylindrical shape to provide a shortened elongate opening, and an offset elongate opening to allow a rolling introduction of the catheter to the guide channel to prevent abrasion of any coatings disposed on the catheter.

Disclosed herein is an insertion tool for introducing a catheter into an introducer including, an attachment structure configured to engage a proximal end of the introducer, and a guide channel axially aligned with a lumen of the introducer when the attachment structure is engaged to the proximal end of the introducer, the guide channel configured to support a portion of the catheter to maintain columnar integrity of the catheter during insertion of the catheter into the introducer.

In some embodiments, the guide channel defines an arcuate cross-section and includes a radius of curvature commensurate with a radius of curvature of an outer surface of the catheter. The guide channel extends through an arc of 30 degrees (30°) or more. The guide channel encircles the portion of the catheter disposed therein. The attachment structure includes a clip that engages an outer surface of a connector disposed at the proximal end of the introducer. The clip includes a first clip arm and a second clip arm that co-operate to at least partially encircle an outer surface of the connector. The attachment structure includes a threaded portion that engages a threaded portion disposed at a proximal end of the introducer. The attachment structure includes an opening and a recess, the recess engages a flange of a connector disposed at the proximal end of the introducer to inhibit longitudinal movement of the connector through the opening.

Also disclosed is a tool for inserting a catheter into an introducer including, a body defining a substantially cylindrical shape, an attachment structure extending distally from a distal end of the body and configured to engage an inner surface of a connector of the introducer, and a guide channel extending from a proximal end of the body to a distal end of the attachment structure, the guide channel defining a pathway for the catheter and configured to maintain columnar integrity of the catheter during insertion of the catheter into the introducer.

In some embodiments, the tool further includes an elongate opening extending longitudinally and extending from the guide channel to an outer surface of the tool. In some embodiments, a lateral width of the elongate opening is less than an outer diameter of the catheter. In some embodiments, a transverse axis of the elongate opening is laterally offset from a central axis of the guide channel. The body further includes a cylindrical segment shape including a horizontal upper surface that provides a transversely shortened elongate opening. In some embodiments, an outer surface of the body defines one of a concave or a bi-concave shape. The body includes a plurality of ribs extending radially therefrom, the plurality of ribs defining an outer perimeter of the body.

In some embodiments, the guide channel defines a tapered shape progressively reducing in diameter from a first diameter at a proximal end, to a second diameter at a distal end. The attachment structure engages the inner surface of the connector in an interference fit, friction fit, press-fit, or snap fit engagement. The attachment structure includes a ridge extending radially outward from an outer surface thereof and configured to engage the inner surface of the connector in a friction fit engagement. The attachment structure includes a beveled tip configured to facilitate engagement with the connector.

In some embodiments, a distal tip of the attachment structure extends to a point that is proximal of a valve disposed within the introducer. In some embodiments, a distal tip of the attachment structure extends to a point that is distal of a valve disposed within the introducer and into a lumen of the introducer, the guide channel defining a pathway for the catheter to be advanced distally of the valve into the lumen of the introducer. In some embodiments, the attachment structure engages the valve to secure the catheter insertion tool to the introducer in a friction fit engagement. The valve is one of a slit valve or a duckbill valve.

Also disclosed is a method of inserting a catheter into a lumen of an introducer including, coupling an insertion tool to a proximal end of the introducer, the insertion tool including an attachment structure and a guide channel, the coupling including axially aligning the guide channel with the lumen of the introducer, inserting a portion of the catheter into the guide channel of the insertion tool, advancing the catheter distally through the guide channel until a distal portion is disposed within the lumen of the introducer, and disengaging the insertion tool from the catheter.

In some embodiments, inserting the catheter into the guide channel of the insertion tool includes inserting the catheter longitudinally into a proximal end of the insertion tool. In some embodiments, inserting the catheter into the guide channel of the insertion tool includes inserting the catheter perpendicular to the longitudinal axis through an elongate opening into the guide channel. In some embodiments, inserting the catheter perpendicular to a longitudinal axis further includes inserting the catheter using a rolling motion against a side wall of the elongate opening.

In some embodiments, disengaging the insertion tool from the catheter further includes withdrawing the insertion tool proximally to disengage the insertion tool from the introducer, prior to disengaging the insertion tool from the catheter in a perpendicular direction to the longitudinal axis of the catheter. In some embodiments, disengaging the insertion tool from the catheter further includes simultaneously disengaging the tool from the introducer and from the catheter in a perpendicular direction to the longitudinal axis of the catheter.

In some embodiments, the coupling further includes an outer surface of the attachment structure engaging an inner surface of a connector of the introducer. In some embodiments, the coupling further includes a ridge disposed an outer surface of the attachment structure engaging an inner surface of a connector of the introducer, a distal tip of the attachment structure extending to a point that is proximal of a valve of the introducer. In some embodiments, the coupling further includes inserting the distal tip of the attachment structure distally past a valve and into the lumen of the introducer. In some embodiments, the coupling further includes the attachment structure engaging an outer surface of a connector that extends proximally from the introducer. In some embodiments, the attachment structure engages the outer surface of the connector in a threaded engagement.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of an exemplary introducer device, in accordance with embodiments disclosed herein.

FIG. 1B shows a close up view of a connector of the introducer device of FIG. 1A, in accordance with embodiments disclosed herein.

FIG. 2A shows a side view of an insertion tool aligned with an exemplary introducer and a catheter, in accordance with embodiments disclosed herein.

FIG. 2B shows a proximal end view of an insertion tool, in accordance with embodiments disclosed herein.

FIG. 2C shows a side view of the insertion tool assembled with the exemplary introducer and the catheter of FIG. 2A, in accordance with embodiments disclosed herein.

FIG. 3A shows a perspective view of an insertion tool, in accordance with embodiments disclosed herein.

FIG. 3B shows a side view of the insertion tool of FIG. 3A, assembled with an exemplary introducer and a catheter, in accordance with embodiments disclosed herein.

FIG. 4A shows a perspective view of an insertion tool, in accordance with embodiments disclosed herein.

FIG. 4B shows a side view of the insertion tool of FIG. 4A, assembled with an exemplary introducer and a catheter, in accordance with embodiments disclosed herein.

FIG. 5A shows a perspective view of an insertion tool, in accordance with embodiments disclosed herein.

FIG. 5B shows a distal end view of an insertion tool, in accordance with embodiments disclosed herein.

FIG. 6A shows a perspective view of an insertion tool, in accordance with embodiments disclosed herein.

FIG. 6B shows a lateral cross-section view of the insertion tool of FIG. 6A, in accordance with embodiments disclosed herein.

FIG. 6C shows a side view of the insertion tool of FIG. 6A, in accordance with embodiments disclosed herein.

FIG. 6D shows a longitudinal cross-section view of the insertion tool of FIG. 6A, in accordance with embodiments disclosed herein.

FIGS. 6E-6G show distal end views of the insertion tool of FIG. 6A and an exemplary catheter, in accordance with embodiments disclosed herein.

FIG. 7A shows a perspective view of an insertion tool, in accordance with embodiments disclosed herein.

FIG. 7B shows a side view of the insertion tool of FIG. 7A, assembled with an exemplary introducer and a catheter, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

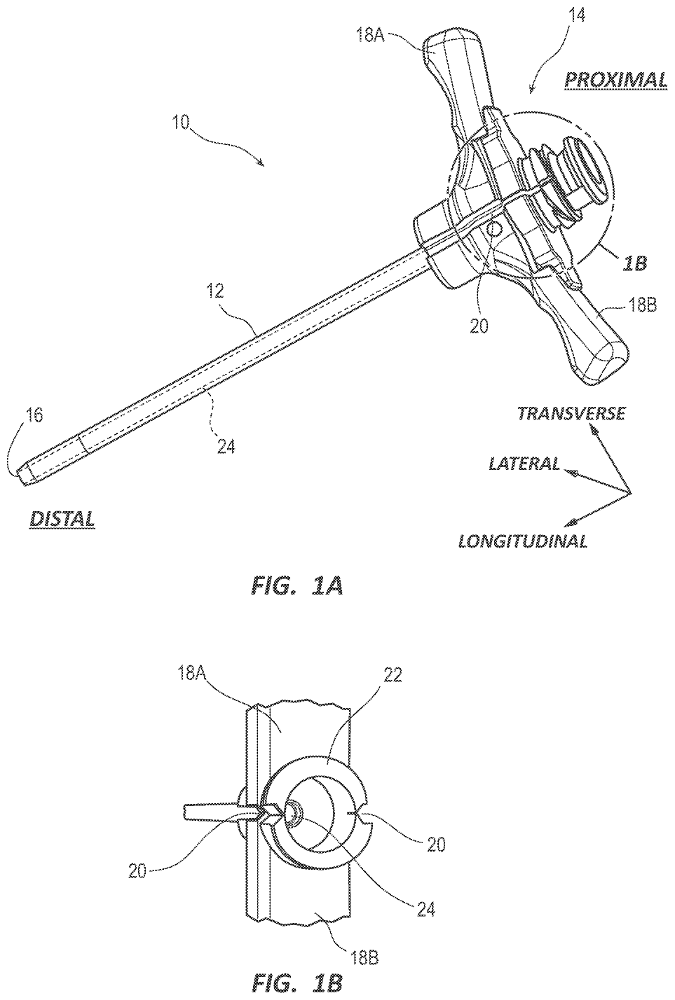

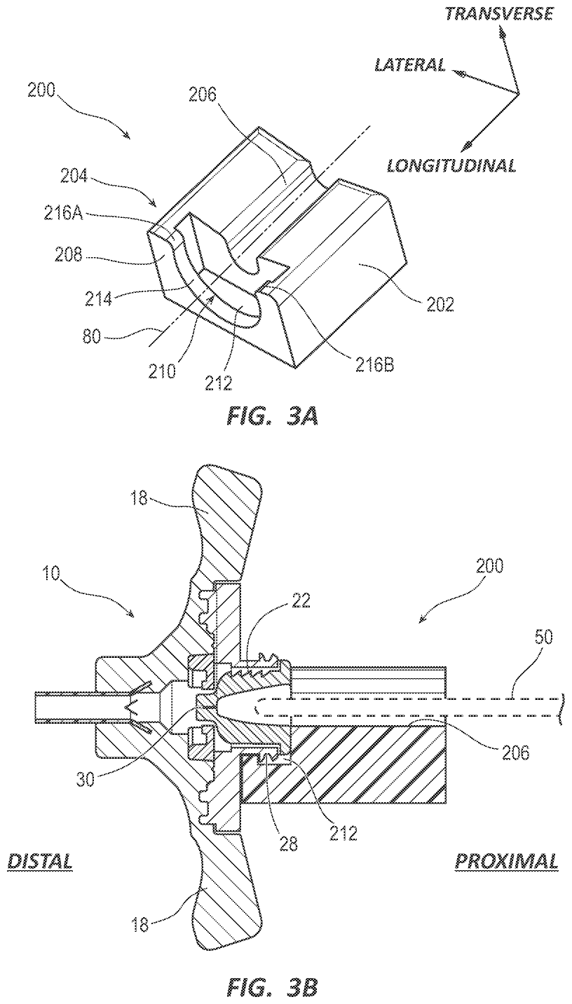

To assist in the description of embodiments described herein, as shown in FIGS. 1A, and 3A, a longitudinal axis extends substantially parallel to an axial length of the introducer shaft 12. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIGS. 1A-1B show various details of an introducer 10. The introducer 10 as shown is exemplary and can include various microintroducers, dilators, splittable introducers, sacrificial catheters, or similar elongate devices that define a lumen that is configured to receive a catheter or similar, secondary elongate medical device therein, which is to be introduced into the vasculature of a patient. The introducer 10 includes an introducer shaft 12 that extends along a longitudinal axis from a proximal end to a distal end, and defines an introducer lumen 24. The introducer shaft 12 includes an introducer hub (“hub”) 14 disposed at the proximal end and an introducer tip (“tip”) 16 disposed the distal end.

The hub 14 includes a first handle 18A and a second handle 18B, each extending from the hub 14, perpendicular to the longitudinal axis. It will be appreciated that the handles 18 can extend laterally, transversely, or at an angle there between, without departing from the spirit of the invention. The hub 14 further includes a breach line 20, extending longitudinally and configured to facilitate separation of the hub 14 and shaft 12, when the handles 18 are urged radially outward from the longitudinal axis. The hub 14 further includes a connector 22 disposed at a proximal end thereof, and is in fluid communication with introducer lumen 24. The connector 22 is configured to couple with additional devices and can include for example a male or female threaded portions, spin nut, luer lock, snap-fit engagements, combinations thereof, or the like.

In an embodiment, the introducer hub 14 includes a valve 30 disposed within the lumen 24 and configured to prevent proximal flow, but does allow catheters, or similar secondary elongate medical devices to be urged distally through the valve 30 and into the lumen 24. Exemplary valves 30 include slit valves, duck-billed valves, flap valves, and the like. It is important to note that due to the flexible properties of the catheter, some catheters buckle or kink when urged against the valve 30, which can prevent the catheter from successfully traversing the valve 30. This is further complicated when the catheter includes, for example, a slit valve disposed at a distal end thereof. As such, the columnar strength of such devices is insufficient to be urged through the valve and results in the catheter kinking and/or collapsing.

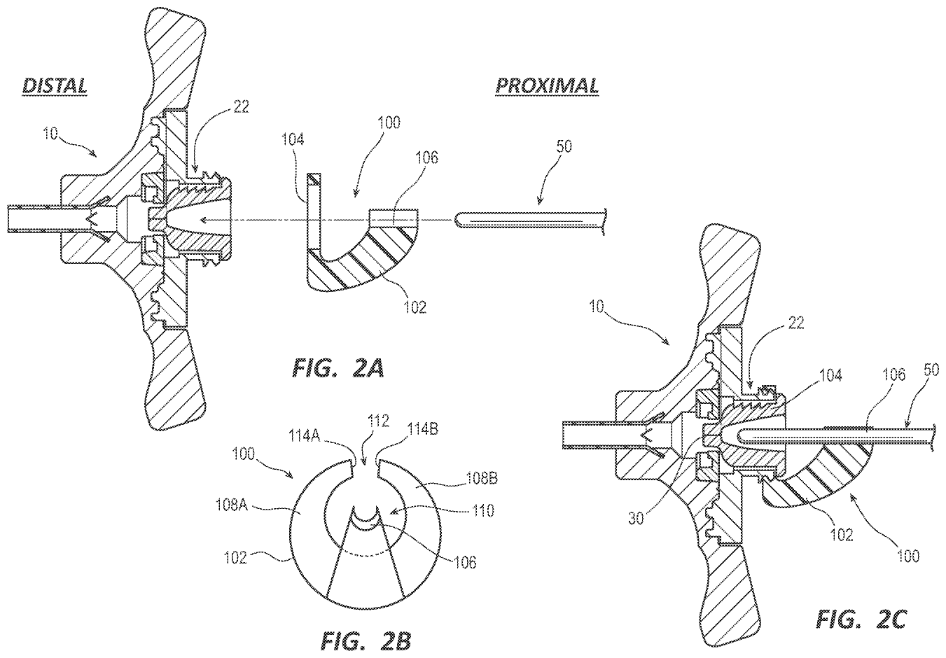

Disclosed herein are embodiments of an insertion tool to enable the introduction of pliable catheters into introducers such as those shown in FIGS. 1A-1B. FIGS. 2A-2C show an embodiment of an insertion tool 100. The insertion tool 100 includes a body 102 including an attachment structure 104 and a guide channel 106. The attachment structure 104 is configured to couple with the introducer hub 14 to releasably secure the tool 100 thereto. In an embodiment, the attachment structure 104 includes a clip, threaded portion, interference fit, snap fit, combinations thereof, or the like, that engages the introducer connector 22.

FIG. 2B shows a proximal end view of the insertion tool 100. In an embodiment, the tool attachment structure 104 includes a clip 108 including a first clip arm 108A and a second clip arm 108B, opposite the first clip arm 108A. The clip arms 108A, 108B are formed of a resilient material that allows the arms 108 to deflect to receive the introducer connector 22 therebetween.

In an embodiment, the first clip arm 108A and the second clip arm 108B define an opening 110 configured to receive a portion of the connector 22 therein. The clip 108 can engage the connector 22 in an interference fit. In an embodiment, the clip arms 108 include a threaded portion on an axial inner surface thereof that engages the threaded connector 22. In an embodiment, the clip arms include various numbers and configurations of barbs, clips, detents, protrusions, or the like that engage the connector 22. In an embodiment, the attachment structure 104 is configured to allow longitudinal ingress of the connector 22 into the opening 110 but inhibit longitudinal egress thereof. As such the connector 22 can be removed perpendicularly, through the passageway 112.

As shown in FIG. 2B, in an embodiment, the first clip arm 108A and the second clip arm 108B extend partially around the circumference of the connector 22 such that the first end surface 114A of the first clip 108A and the second end surface 114B of the second clip 108B to define a passageway 112 there between. In an embodiment, the first clip arm 108A and the second clip arm 108B extend circumferentially around the entire connector 22 such that a first end surface 114A of the first clip 108A abuts against a second end surface 114B of the second clip 108B to define a slit there between. As noted the clip arms 108A, 108B, are formed of a resilient material that allows some deflection of the clips arms. Accordingly, the passageway 112, or slit, allows the clips arms 108A, 108B to move relative to each other. In an embodiment the connector 22 can be urged longitudinally into the opening 110. In an embodiment, the passageway 112 allows ingress and egress of the connector 22 into/out of the opening 110, perpendicular to the longitudinal axis.

In an embodiment, the insertion tool 100 includes a guide channel 106 that defines an arcuate cross sectional shape. The radius of curvature of the cross-sectional shape of the channel 106 is configured to substantially match the radius of curvature of the outer surface of the catheter 50. The guide channel 106 is configured to support the portion of the catheter 50 disposed therein to maintain columnar integrity of the catheter 50, preventing kinking or collapsing of the catheter 50 as it is urged distally. In an embodiment, the cross-section of the guide channel 106 extends through an arc of 30° or more. In an embodiment the cross-section of the guide channel 106 extends through an arc of 180°. In an embodiment, the guide channel 106 defines a closed channel that completely encircles a portion of the catheter 50 disposed therein. A central axis of the channel 106 aligns with a central axis of the opening 110. As such, when the insertion tool 100 is coupled with the introducer 10 a central axis of the guide channel 106 aligns with the central axis of the connector 22 and introducer lumen 24.

In an exemplary method of use, a distal tip 16 of an introducer 10 is placed within the vasculature of the patient to provide access thereto. An insertion tool 100 is coupled with the connector 22 by urging the connector 22 longitudinally into the opening 110. Optionally, the connector 22 can be urged transversely or laterally through passageway 112 into the opening 110. A distal portion of catheter 50 is disposed within guide channel 106 that aligns the catheter 50 with the lumen 24 of the introducer. As described herein, the guide channel 106 provides columnar support for the catheter 50 so as to maintain columnar integrity of the catheter 50, preventing kinking or collapsing. The catheter 50 can then be slid distally through the guide channel 106 until a distal tip thereof engages the valve 30. The catheter 50 is then urged distally past the valve 30 with the guide channel 106 providing columnar support to the catheter 50 to prevent kinking or collapsing. Optionally, a clinician can rest a digit on the portion of the catheter 50 disposed within the channel to support the catheter 50 against the guide channel 106 and provide additional columnar support.

FIGS. 3A-3B show an embodiment of an insertion tool 200. The insertion tool 200 includes a tool body 202, an attachment structure 204, and a guide channel 206. The attachment structure 204 is configured to couple with the introducer connector 22 and to align a central axis of the guide channel 206 with the central axis of the introducer 10, as described herein.

In an embodiment, the insertion tool 200 defines a substantially cuboid shape, including a substantially flat distal surface extending perpendicular to the longitudinal axis. The attachment structure 204 is disposed at a distal end of the tool 200 and is configured to retain a portion of the introducer connector 22 therein. In an embodiment, the attachment structure 204 includes a substantially U-shaped notch (“notch”) 210, disposed in a distal surface 208 of the tool 200, which communicates with a cuboid shaped recess 212.

In an embodiment, the notch 210 defines a radius of curvature that substantially matches the radius of curvature of the outer surface of the introducer connector 22. In an embodiment, a perimeter surface 214 of the notch 210 extends through an arc of 30° or more. In an embodiment, a perimeter surface 214 of the notch 210 extends through an arc of 180° or more. In an embodiment, a perimeter surface 214 defines a first protrusion 216A and a second protrusion 216B disposed laterally opposite each other across a central axis 80. A lateral distance between the first protrusion 216A and the second protrusion 216B is slightly less than a diameter of the introducer connector 22. As such as a portion of the introducer connector 22 is urged transversely downward into the tool attachment structure 204, the protrusions 216A, 216B co-operate to secure the introducer connector 22 within the notch 210 in a snap-fit or an interference fit engagement.

In an embodiment, the introducer connector 22 further includes a flange structure (“flange”) 28 extending radially from an outer surface thereof. Exemplary flange structures can include a threaded portion, protrusion, lugs, bayonet connector, twist lock, or similar structure that can extend annularly about at least a portion of the connector 22. The outer diameter of the flange 28 is larger than an inner diameter of the notch 210, but less than a lateral width of the recess 212. Accordingly, as the connector 22 is urged transversely downwards into the notch 210, the recess 212 is configured to receive the flange 28. The flange engages an inner surface of the recess 212 and inhibits the connector 22 from being axially withdrawn from the notch 210.

In an embodiment, the insertion tool 200 includes a guide channel 206 that defines an arcuate cross section. The radius of curvature of the channel 206 is commensurate with the radius of curvature of the outer surface of the catheter 50. The guide channel 206 is configured to support the portion of the catheter 50 disposed therein to maintain columnar integrity of the catheter 50, preventing kinking or collapsing of the catheter 50 as it is urged distally. In an embodiment, a cross-section of the guide channel 206 extends through an arc of 30° or more. In an embodiment, a cross-section of the guide channel 206 extends through an arc of 180°. A central axis of the channel 206 aligns with a central axis of the notch 210. As such, when the insertion tool 200 is coupled with the introducer 10 a central axis of the guide channel 206 aligns with the central axis of the connector 22 and introducer lumen 24.

In an exemplary method of use, a distal tip 16 of an introducer 10 is placed within the vasculature of the patient to provide access thereto. An insertion tool 200 is coupled with the connector 22 by urging the connector 22 transversely downward into the attachment structure 204. A portion of the connector is received within the notch 210 with the protrusions 216A, 216B co-operating to retain the connector therein. The flange 28 is received within the recess 212. A distal portion of catheter 50 is disposed within guide channel 206 that aligns the catheter 50 with the lumen 24 of the introducer and provides columnar support for the catheter 50. The catheter 50 can then be slid distally through the guide channel 206 until a distal tip thereof engages the valve 30. The catheter 50 is then urged distally past the valve 30 with the guide channel 206 providing columnar support to the catheter 50 to prevent kinking or collapsing. Optionally, a clinician can rest a digit on the portion of the catheter 50 disposed within the channel to support the catheter 50 against the guide channel 206 and provide additional columnar support. In an embodiment, an upper surface of the insertion tool 200 can include one or more angled surfaces to facilitate directing the portion of catheter 50 towards the guide channel 206.

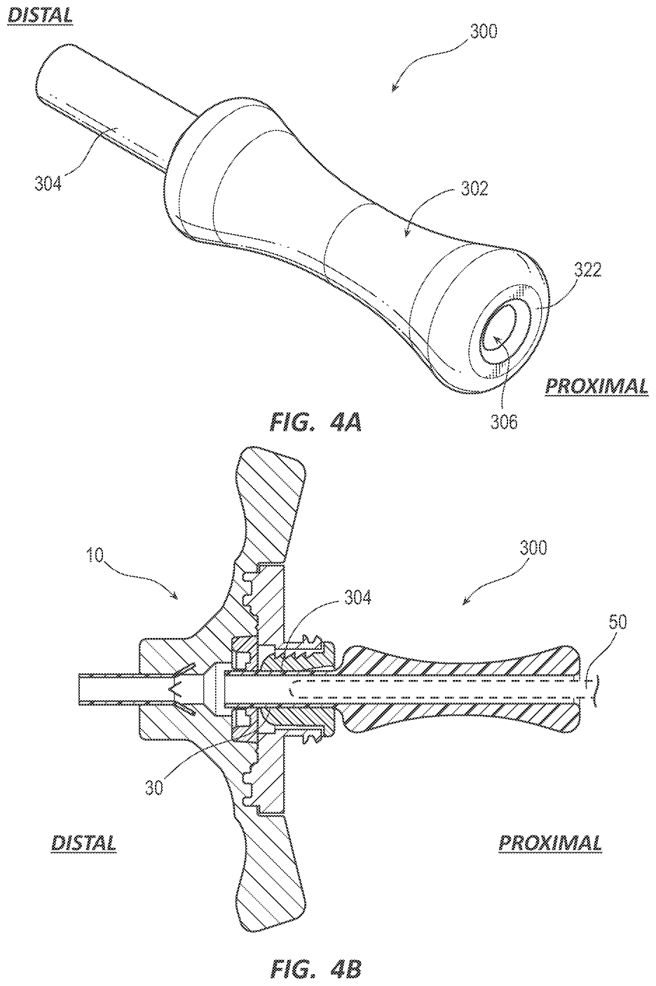

FIGS. 4A-4B show an embodiment of an insertion tool 300, including a tool body 302 defining a guide channel 306 and an attachment structure 304 extending distally from a distal surface of the tool body 302. The tool body 302 defines a substantially cylindrical shape including a circular cross section. As shown in FIG. 4B, the tool body 302 further includes a concave outer side surface configured to direct a user to grasp a mid-point of the tool body 302.

The guide channel 306 extends from a proximal end of the tool body 302 to a distal end of the attachment structure 304, and is configured to receive a portion of the catheter 50 therein. In an embodiment, the inner diameter of the guide channel 306 is substantially the same or slightly larger than the outer diameter of the catheter 50 so that the catheter 50 fits snugly therein. Further, the proximal end of the guide channel 306 can include a chamfered edge 322 to facilitate introduction of the catheter 50 into the guide channel 306. As shown in FIG. 4B, the attachment structure 304 defines an outer diameter that is less than the inner diameter of the connector 22 and valve 30. The attachment structure 304 defines an axial length that allows a distal tip of the attachment structure 304 to be inserted past the valve 30, defining a pathway for the catheter 50 to be inserted into the introducer lumen 24.

In an exemplary method of use, a distal tip 16 of an introducer 10 is placed within the vasculature of the patient to provide access thereto. An insertion tool 300 is coupled with the connector 22 by urging the attachment structure 304 longitudinally into the connector 22. In an embodiment, the distal tip of the attachment structure 304 can engage an inner surface of the connector 22 to secure the insertion tool 300 with the introducer 10. The attachment structure 304 can engage the introducer 10 in one of an interference fit, friction fit, press-fit, or snap fit engagement. In an embodiment, a distal tip of the attachment structure 304 can extend distally, past the valve 30. The friction between the attachment structure 304 engaging the valve 30, lumen 24, or combinations thereof, can secure the insertion tool 300 to the introducer 10. Further the attachment structure 304 opens the valve 30 to provide an unobstructed pathway for the catheter 50 to pass into the introducer lumen 24.

A distal portion of catheter 50 is disposed within a proximal portion of the guide channel 306. In an embodiment, the catheter 50 is disposed within the guide channel 306 after the attachment structure 304 is inserted into the introducer lumen 24. In an embodiment, the catheter 50 is “preloaded” within the guide channel 306, i.e., disposed within the guide channel 306 before the attachment structure 304 is inserted into the introducer lumen 24. As previously noted, the catheter 50 may provide a snug fit within the guide channel 306 to prevent or limit proximal flow from the introducer 10 when the valve 30 is opened. Further, the snug fit of the catheter 50 within the guide channel 306 provides columnar support for the catheter 50 as it is urged distally, preventing kinking or collapsing. Once a distal portion of the catheter 50 is disposed within the introducer lumen 24, the insertion tool 300 can be withdrawn proximally to disengage the attachment structure 304 from the introducer, and withdrawn from the catheter proximally.

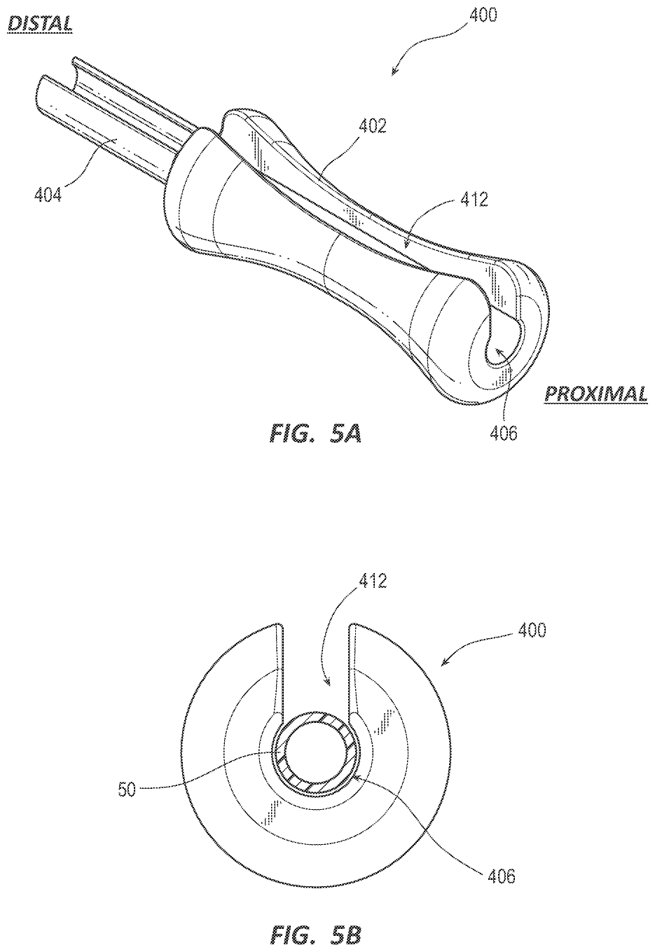

FIGS. 5A-5B show an embodiment of an insertion tool 400, including a tool body 402 defining a guide channel 406 and an attachment structure 404 extending distally from a distal surface of the tool body 402. As described herein, the tool body 402 defines a substantially cylindrical shape including a circular cross section and further includes a concave outer side surface. The guide channel 406 extends from a proximal end of the tool body 402 to a distal end of the attachment structure 404, and is configured to receive a portion of the catheter 50 therein. In an embodiment, the inner diameter of the guide channel 406 is substantially the same or slightly larger than the outer diameter of the catheter 50 so that the catheter 50 fits snugly therein.

The insertion tool 400 further includes an elongate opening 412, extending longitudinally, and extending from the guide channel 406 to an outer surface of the insertion tool 400. The elongate opening is configured to allow ingress or egress of the catheter 50 from the channel 406, perpendicular to the longitudinal axis. As shown in FIG. 5B, a lateral width of the elongate opening 412 is less than an outer diameter of the catheter 50. As such, the catheter 50 deforms slightly in order to pass through the elongate opening, and is then allowed to resume its undeformed shape when seated in the guide channel 406. Similarly during egress, the catheter 50 must deform slightly to be urged through the elongate opening 412. In some embodiments, the catheter 50, the elongate opening 412, or combinations thereof, deform slightly to allow the catheter 50 to pass through the elongate opening 412, into or out of the channel 406. Advantageously, the lateral width of the elongate opening, configured as such, retains the portion of the catheter 50 within the channel 406. Further, the columnar strength of the catheter 50 is maintained during insertion as it is urged distally.

In an exemplary method of use, the attachment structure 404 engages the connector 22 and valve 30. The catheter 50 is inserted into a proximal end of the channel 406 and advanced past the valve 30 and into the introducer lumen 24, as described herein. The insertion tool 400 can be removed from the introducer 10 by withdrawing the tool 400 proximally, disengaging the attachment structure 404 from the valve 30 and introducer connector 22. The tool 400 can then be removed from the catheter 50 by urging the catheter perpendicular to the longitudinal axis, through the elongate opening 412. Advantageously, the insertion tool 400 can be removed from the catheter 50 when proximal features, such as catheter hubs, and the like, inhibit proximal removal of the insertion tool from the catheter 50.

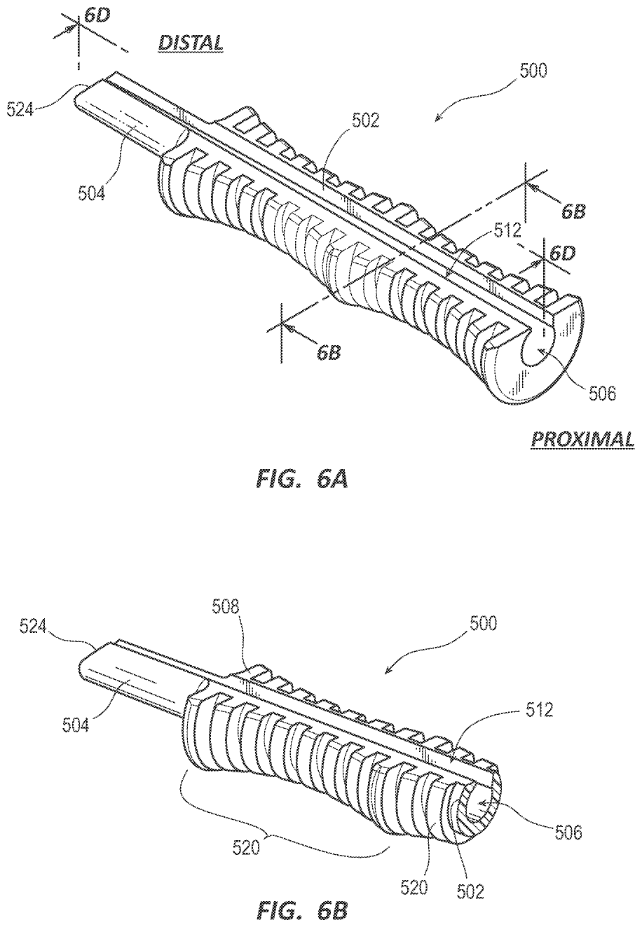

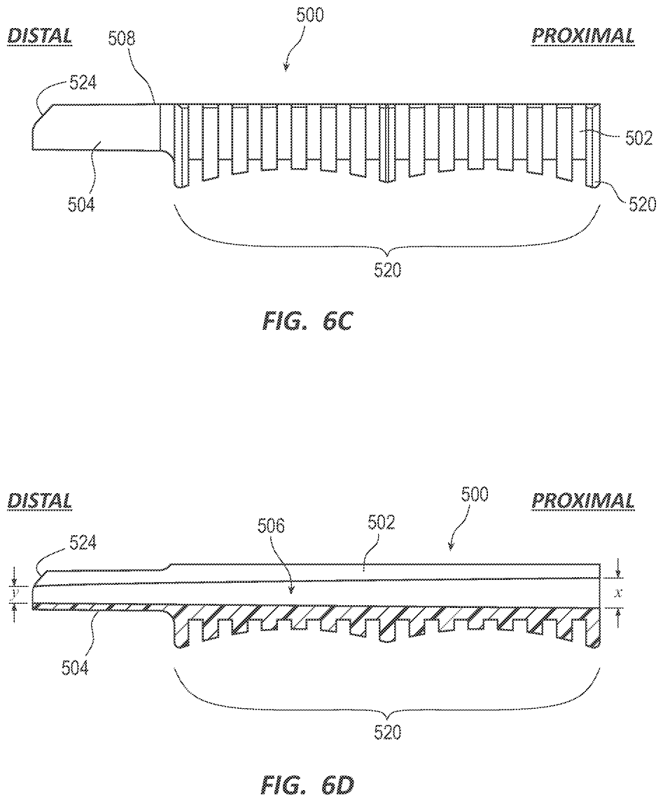

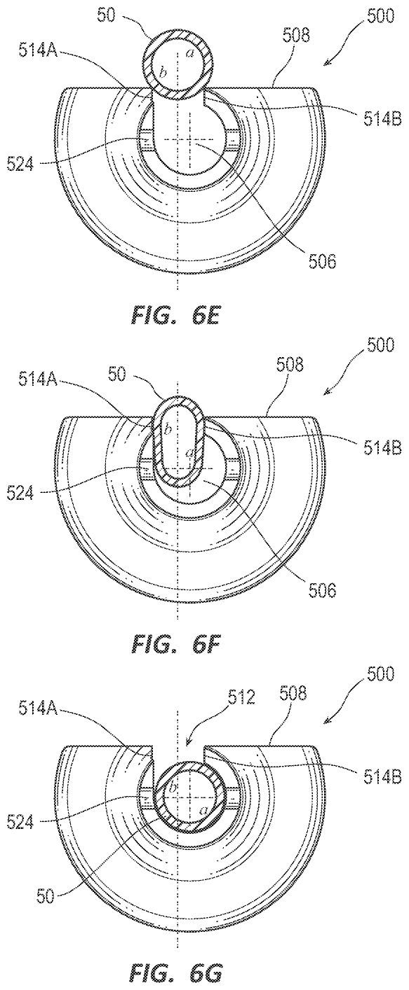

FIGS. 6A-6G show an embodiment of an insertion tool 500, including a tool body 502 defining a guide channel 506 and an attachment structure 504 extending distally from a distal surface of the tool body 502. A distal end of the attachment structure 504 includes one or more chamfered edges, beveled tips, or combinations thereof, e.g. beveled tip 524, to facilitate insertion of the distal end of the attachment structure 504 past the valve 30 without damaging the valve. The tool body 502 defines a substantially horizontal cylindrical segment shape with a cylindrical lower portion and a flat, horizontal upper surface 508. FIG. 6B shows a lateral cross-sectional view of the insertion tool 500 of FIG. 6A, and FIG. 6C shows a side view of the insertion tool 500 of FIG. 6A. As shown, the tool body 502 further includes a plurality of ribs 520 extending radially from an outer surface of the tool body 502. In an embodiment, each of the ribs 520 extend equally from the tool body 502 to define a substantially cylindrical outer perimeter. In an embodiment, the ribs 520 extend at different distances from the tool body 502 to define a substantially concave outer perimeter, as described herein. In an embodiment, as shown in FIG. 6C, the ribs 520 extend radially to define a bi-concave outer perimeter. The bi-concave outer profile guides a user to grasp either a first position or a second position along the outer surface of the insertion tool 500. Advantageously, the ribs 520 also provide improved grip for the user grasping the tool 500, as well as reducing the amount of material required to form the tool 500, reducing costs while maintaining structural integrity.

FIG. 6D shows a longitudinal cross-section view of the insertion tool 500 of FIG. 6A. FIGS. 6E-6G show a distal end view of the insertion tool 500 of FIG. 6A. The guide channel 506 extends from a proximal end of the tool body 502 to a distal end of the attachment structure 504, and is configured to receive a portion of the catheter 50 therein. In an embodiment, the guide channel 506 defines a tapered shape, wherein an inner diameter of the proximal end of the guide channel 506 defines a first diameter (x) and an inner diameter of the distal end defines a second diameter (y). The second diameter (y) is smaller than the first diameter (x) and the channel 506, extending therebetween, progressively reduces from the first diameter (x) to the second diameter (y). In an embodiment, the first diameter (x) defines an opening that is larger than the outer diameter of the catheter 50 to facilitate insertion of the catheter 50 into the guide channel 506. In an embodiment, the proximal end of the channel 506 can include a chamfered entrance to further facilitate insertion of the catheter 50 into the guide channel 506. In an embodiment, the second diameter (y) is substantially the same, or slightly smaller than the outer diameter of the catheter 50 so that the catheter 50 fits snugly therein. Advantageously, the catheter 50 can be easily inserted proximally into the guide channel, then as the catheter 50 is advanced towards the distal end, the diameter of the channel 506 progressively reduces to the second diameter (y), providing increased columnar support.

As shown in FIGS. 6A-B and 6E-6G, the insertion tool 500 further includes an elongate opening 512, extending longitudinally, and extending from the guide channel 506 to an outer surface of the insertion tool 500. The elongate opening is configured to allow ingress or egress of the catheter 50 from the channel 506, perpendicular to the longitudinal axis. The segmented cylindrical cross-section of the insertion tool 500 provides a shortened elongate opening 512 through which the catheter 50 passes. The shortened elongate opening 512 reduces that amount of friction between the catheter 50 and the side walls 514 of the elongate opening during ingress and egress. This in turn reduces wear on the outer surface of the catheter 50 that can remove any coatings disposed thereon, reducing the efficacy of the coating.

As shown in FIGS. 6E-6G, a lateral width of the elongate opening 512 is less than an outer diameter of the catheter 50, to retain the portion of catheter 50 within the channel 506. As such, as shown in FIG. 6F, the catheter 50 deforms slightly in order to pass through the elongate opening, and is then allowed to resume its undeformed shape when seated in the guide channel 506, as shown in FIG. 6G. Similarly during egress, the catheter 50 deforms slightly to be urged through the elongate opening 512. This facilitates retention of the catheter 50 within the guide channel 506, as described herein.

Also shown in FIGS. 6E-6G, a transverse axis of the elongate opening 512 is laterally offset from a central axis of the guide channel 506. This allows the catheter 50 to be inserted into the channel 506, using a rolling motion against a first side wall 514A of the elongate opening 512. For example, as shown in FIG. 6E, during ingress of the catheter 50 to the channel 506, the catheter 50 can be placed at the entrance of the elongate opening 512. As shown in FIG. 6F, a side wall of the catheter 50 at position (a), can be deformed inwards and downwards, past the second side wall 514B of the elongate opening 512. As such, the opposite side wall of the catheter 50 at position (b) rolls down the first side wall 514A of elongate opening 512. As shown in FIG. 6G, the catheter 50 then resumes its original undeformed shape within the guide channel 506.

Advantageously, the offset configuration of the elongate opening 512 allows for the rolling motion of ingress and egress of the catheter to/from the channel 506. This in turn prevents abrading the catheter against the side walls 514A, 514B of the elongate opening, which can remove any coatings disposed thereon, e.g. lubricious coatings, anti-microbial coatings, etc.

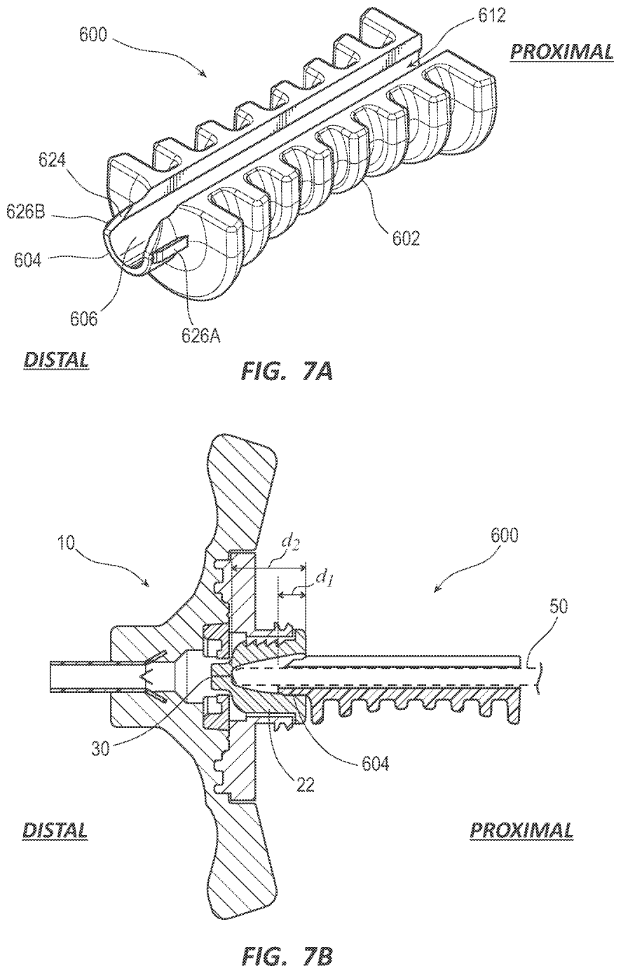

FIGS. 7A-7B show an embodiment of a catheter insertion tool 600 including a tool body 602 defining a guide channel 606 and an attachment structure 604 extending distally from a distal surface of the tool body 602. The tool body 602 includes an elongate longitudinal opening 612 allowing ingress/egress of the catheter 50 to/from the channel 606, as described herein. A distal end of the attachment structure 604 includes one or more chamfered edges, beveled tips, or combinations thereof, e.g. beveled tip 624, to facilitate engagement of the distal end of the attachment structure 604 with the connector 22.

In an embodiment, a longitudinal length (d1) of the attachment structure 604 can be less than a longitudinal length (d2) between a proximal surface of the introducer hub 14 and the valve 30. As such, the attachment structure 604 can engage an inner surface of the connector 22 in an interference fit but does not interact with the valve 30. Worded differently, a distal tip of the attachment structure 604 can extend to a point that is proximal and adjacent to the valve 30 but does not interact with the valve 30 when the tool 600 engages the connector 22. Advantageously, the attachment structure 604 can secure the insertion tool 600 to the introducer 10 while still allowing the valve to 30 control a proximal fluid flow. Further, the tool 600 can provide columnar support to the catheter 50 up to the point of interaction with the valve 30, thereby mitigating any kinking or buckling of the catheter 50 between the attachment structure 604 and the valve 30.

In an embodiment, the attachment structure 604 includes a ridge 626 extending radially outward from the outer surface of the attachment structure 604 and extending longitudinally. The attachment structure 604 can include a first ridge 626A disposed on a first side of the attachment structure 604, and a second ridge 626B disposed on a second side of the attachment structure 604 opposite the first side, across the longitudinal axis. The ridge(s) 626 can be formed of a resilient material and can engage an inner surface of the connector 22 to provide an interference fit engagement, or friction fit engagement, between the attachment structure 604 and the introducer 10. It will be appreciated that the attachment structure 604 can include various numbers or configurations of ridges 626 without departing from the spirit of the invention. In an embodiment, the attachment structure 604 can include one or more protrusions, detents, barbed structures or the like, extending therefrom and configured to engage the inner surface of the connector in a snap-fit engagement.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An insertion tool for introducing a catheter into an introducer, comprising:
an attachment structure configured to engage a proximal end of the introducer, a distal tip of the attachment structure extending to a point that is proximal of a valve disposed within a lumen of the introducer; and
a guide channel axially aligned with the lumen of the introducer when the attachment structure is engaged to the proximal end of the introducer, the guide channel configured to support a portion of the catheter to maintain columnar integrity of the catheter during insertion of the catheter into the introducer.

2. The insertion tool according to claim 1, wherein the guide channel defines an arcuate cross-section and includes a radius of curvature commensurate with a radius of curvature of an outer surface of the catheter.

3. The insertion tool according to claim 2, wherein the guide channel extends through an arc of 30 degrees (30°) or more.

4. The insertion tool according to claim 2, wherein the guide channel encircles the portion of the catheter disposed therein.

5-8. (canceled)

9. A tool for inserting a catheter into an introducer, comprising:
a body defining a substantially cylindrical shape;
an attachment structure extending distally from a distal end of the body and configured to engage an inner surface of a connector of the introducer, a distal tip of the attachment structure extends to a point that is proximal of a valve disposed within the introducer when the tool is engaged with the introducer; and
a guide channel extending from a proximal end of the body to the distal tip of the attachment structure, the guide channel defining a pathway for the catheter and configured to maintain columnar integrity of the catheter during insertion of the catheter into the introducer.

10. The catheter insertion tool according to claim 9, further including an elongate opening extending longitudinally and extending from the guide channel to an outer surface of the tool.

11. The catheter insertion tool according to claim 10, wherein a lateral width of the elongate opening is less than an outer diameter of the catheter.

12. The catheter insertion tool according to claim 10, wherein a transverse axis of the elongate opening is laterally offset from a central axis of the guide channel.

13. The catheter insertion tool according to claim 10, wherein the body further includes a cylindrical segment shape including a horizontal upper surface that provides a transversely shortened elongate opening.

14. The catheter insertion tool according to claim 9, wherein an outer surface of the body defines one of a concave or a bi-concave shape.

15. The catheter insertion tool according to claim 9, wherein the body includes a plurality of ribs extending radially therefrom, the plurality of ribs defining an outer perimeter of the body.

16. The catheter insertion tool according to claim 9, wherein the guide channel defines a tapered shape progressively reducing in diameter from a first diameter at a proximal end of the guide channel, to a second diameter at a distal end of the guide channel.

17. The catheter insertion tool according to claim 9, wherein the attachment structure engages the inner surface of the connector in an interference fit, friction fit, press-fit, or snap fit engagement.

18. The catheter insertion tool according to claim 9, wherein the attachment structure includes a ridge extending radially outward from an outer surface thereof and configured to engage the inner surface of the connector in a friction fit engagement.

19. The catheter insertion tool according to claim 9, wherein the attachment structure includes a beveled tip configured to facilitate engagement with the connector.

20. The catheter insertion tool according to claim 9, wherein a longitudinal length of the attachment structure is less than a longitudinal length between a proximal surface of a hub of the introducer and the valve.

21-22. (canceled)

23. The catheter insertion tool according to claim 20, wherein the valve is one of a slit valve or a duckbill valve.

24-34. (canceled)

35. The catheter insertion tool according to claim 1, further including an elongate opening extending longitudinally and extending from the guide channel to an outer surface of the tool.

36. The catheter insertion tool according to claim 1, wherein the attachment structure engages the inner surface of the connector in an interference fit, friction fit, press-fit, or snap fit engagement.

37. The catheter insertion tool according to claim 1, wherein the attachment structure includes a ridge extending radially outward from an outer surface thereof and configured to engage the inner surface of the connector in a friction fit engagement.
